(19) United States
(12) Patent Application Publication
(10) Pub. No.: US 2008/0097786 A1
(43) Pub. Date: Apr. 24, 2008

(54) Digital data security in healthcare enterprise

(76) Inventor: Rohit Sachdeva, Plano, TX (US)

Correspondence Address: Memory Medical Systems, 2605 Courtside Lane, Plano, TX, 75093, US

(21) Appl. No.: 11/582,764

(22) Filed: Oct. 18, 2006

(51) Int. Cl. G06Q 10/00; G06F 19/00; G06F 17/30; G06Q 50/00; A61B 5/00; G06F 7/04; G06K 9/00; H03M 1/68; H04K 1/00; H04L 9/00; H04L 9/32; H04N 7/16

(52) U.S. Cl. 705/2; 726/30; 705/3

(57) ABSTRACT

Method and system for providing services in healthcare enterprises for authentication and forensic analysis of medical and dental records of patients comprising text, image, video and speech stored and communicated in digital form are disclosed. The digital images may be two-dimensional or three-dimensional comprising, for example, photographs, x-rays, CT-scans, other types of scanned images, video, etc. The patient records include living wills. Patient's original record, record ID, activity log and signature are stored in digital storage along with the original patient record attributes. Access of the records is provided to the users. New versions of record are saved along with the activity log and signature; and attributes of new versions of the record are computes and compared with the original version. If new attributes are not the same as the original attributes, then further analyses is conducted to identify the extent and the nature of record modifications and the source of modifications. The modifications are evaluated to determine if tempering has occurred. This and many other aspects of the invention are disclosed.

### BACKGROUND OF THE INVENTION

A. Field of the Invention

This invention pertains, in a broad sense, to digital data security in healthcare enterprise; and in particular, to services for authentication and forensic analysis of medical and dental records comprising text, image, video and speech stored and communicated in digital form.

B. Description of Related Art

It is increasingly becoming common in healthcare enterprises to store and communicate patient records in digital form. The records comprise information concerning patients in text, image, video and speech forms. Text records include, for example, patient's diagnosis, treatment plan, prescription, billing, insurance and other information. Images may be two-dimensional or three-dimensional; and include, for example, photographs, x-rays, CT-scans, and a variety of other images. In some instances records are created and communicated in the form of digital videos. Speech includes patient's diagnosis, treatment plan, prescription, instructions to other healthcare professionals, etc. These records are accessed by a multitude of professionals and personnel in the healthcare industry, including off-shore practitioners, in order to deliver treatment and care to patients. It is extremely vital that integrity of such digital data be strictly maintained; and any tempering with the records be promptly detected. There is some prior art in the area of watermarking and copywriting digital images; however the art is lacking in the area of comprehensive authentication and forensic analysis services for medical and dental records comprising text, images, video and speech. Frequently, hash code computations are performed prior to transmitting and up-on receiving a record; and compared to detect errors introduced during transmission. While this is a very effective method for detecting transmission errors, it lacks in providing comprehensive authentication and forensic analysis capabilities. For example, a record may tempered and then transmitted from one facility to another; and in the absence of any transmission errors, the hash code corresponding to the record at the transmitting facility will be same as the hash code at the receiving facility, and the record tempering would go undetected. Given the extreme importance of the health care records, an urgent need exists for comprehensive services for authentication and forensic analysis of medical and dental records of all types such as images, video, text and speech stored and communicated in digital form. Given the extremely large volume of such records routinely generated at any typical healthcare provider, it is imperative that the authentication and forensic analysis services be very efficient and cost-effective.

The instant invention disclosed herein provides a method and system for providing comprehensive services for authentication and forensic analysis of medical and dental records comprising images, video, text and speech stored and communicated in digital form.

### SUMMARY OF THE INVENTION

In the first aspect of the invention, a method is disclosed for providing service for authentication of digital records in healthcare enterprises. The digital records may be in the form of text, images, video or speech. The digital images may be two-dimensional or three-dimensional comprising, for example, photographs, x-rays, CT-scans, other types of scanned images, video, etc. The authentication service method comprises the following steps:

a) Prepare patient's original record, record ID, activity log and signature;

b) Transmit patient record, record ID, activity log and signature to Data Security Provider;

c) Compute original patient record attributes;

d) Store patient record, record ID, activity log, signature and attributes in digital storage;

e) Store back-up copy of patient record, record ID, activity log, signature and attributes in digital storage;

f) Provide patient record access to a user;

g) Save new version of record, activity log and signature;

h) Compute and save attributes of new version of record;

i) If new attributes are the same as the original attributes, then stop; otherwise proceed to the next step;

j) Perform further analysis; and report findings.

In another aspect of the invention, a method for record authentication is disclosed comprising the following steps.
a) Prepare & store original patient record, record ID, activity log and signature; Compute original patient record attributes values;
b) Transmit patient record ID, activity log, signature and original patient record attributes values to Data Security Provider;
c) Store original patient record ID, activity log, signature and attributes values in digital storage;
d) Provide patient record access to a user;
e) Save new version of record, activity log and signature;
f) Compute and save attributes values of new version of record;
g) Transmit patient record ID, activity log, signature and attributes values of new version of record to Data Security Provider;
h) Are new attributes values same as original values? If yes, then stop; otherwise proceed to the next step;
i) Perform further analysis; and report findings.

According to one embodiment of the invention, the data security provider provides centralized digital storage devices and facility for digitally storing all versions of the patient records and associated information discussed above such as the record ID, the activity log, the signature and the computed values of the one or more preferred attributes. The records and the information is archived after a certain time period, and preserved for another certain time period mutually agreed upon between the health care provider and the data security provider.

According to another embodiment of the invention, the data security provider provides distributed digital storage devices and facility for digitally storing all versions of the patient records and associated information discussed above. One skilled in the art would appreciate that a combination of the centralized storage for some applications, and the distributed storage for others can very well be realized.

In another aspect of the invention, although the preceding discussion focused on comparing the original version of a patient record with a subsequent version of the patient record; one skilled in the art would appreciate that the method can be generalized for comparing any two versions of the record.

In another aspect of the invention, the authentication process disclosed herein can be applied while facilitating peer-to-peer communication, such as between specialists concerning patients.

In another aspect of the invention, the records may include a patient's living will which may be accessed and shared by appropriate healthcare professionals associated with the treatment of the patient and relatives of the patient.

In yet another aspect of the invention, one skilled in the art would appreciate that in the absence of a verifiable original record, the original record may constitute the record that is authenticated to be the original record by one or more authentication algorithms.

In yet another aspect of the invention, the method can similarly be applied for authentication of any type of records, and in any type of business; such as for example financial records in financial institutions.

In yet another aspect of the invention, the method can be applied for authentication of books and manuscripts or pieces of art kept in the digital form.

In another aspect of the invention, the authentication process disclosed herein can be modified for conducting a forensic analysis in the event that a patient record has been determined to be tempered with in an unjustifiable manner. Since all versions of the records and the information associated with the records are digitally stored and maintained between the health care provider and the data security provider; the entire history of the records can be traced in support of the forensic analysis. In summary, the forensic analysis comprises:

a) digitally storing all versions of records;

b) examining record history and records;

c) identify record changes, location and time; and

d) evaluating the record modifications and identifying the sources.

In yet another aspect of the invention, the method can be used for authenticating, for example, the prescribed medicine being carried by an airline passenger. The authentication process would work as follows:
(a) An official at the airport scans the prescription label on the medicine bottle being carried by a passenger, along with a picture identification of the passenger; and transmits the information digitally to a data security provider;
(b) The data security provider maintains, and continually updates, a database of the valid prescriptions and the picture identifications of the patients;
(c) The data security provider compares the information received from the airport official with the information in the database; and
(d) (i) If the information completely matches, then the data security provider sends a ‘green signal’ to the air port official;
   (ii) If the information matches partially, then the data security provider sends a ‘yellow signal’ to the air port official; suggesting that further investigation may be required; and
   (iii) If the information does not match at all, then the data security provider sends a ‘red signal’ to the air port official; suggesting that the authentication has failed.

In another aspect, a system for authentication of digital records is disclosed. The system comprises one or more workstations, each having a processor and a storage device which stores patient records in digital form in healthcare enterprises. The digital records may be in the form of text, images, video or speech. The digital images may be two-dimensional or three-dimensional comprising, for example, photographs, x-rays, CT-scans, other types of scanned images, video, etc. Additionally, the system provides computer instructions, which are stored in one or more digital storage devices enabling the user to:

a) prepare or receive patient's original record, record ID, activity log and signature;

b) transmit patient record, record ID, activity log and signature to a data security provider;

c) compute original patient record attributes;

d) store patient record, record ID, activity log, signature and attributes in digital storage;

e) store back-up copy of patient record, record ID, activity log, signature and attributes in digital storage;

f) provide patient record access to a user;

g) save new version of record, activity log and signature;

h) compute and save attributes of new version of record;

i) if new attributes are the same as the original attributes, then stop; otherwise proceed to the next step;

j) perform further analysis; and report findings.

In another aspect of the invention, a system for record authentication is disclosed comprising one or more workstations, each having a processor and a storage device which stores patient records in digital form in healthcare enterprises. The digital records may be in the form of text, images, video or speech. The digital images may be two-dimensional or three-dimensional comprising, for example, photographs, x-rays, CT-scans, other types of scanned images, video, etc. Additionally, the system provides computer instructions, which are stored in one or more digital storage devices enabling the user to:
a) prepare or receive & store original patient record, record ID, activity log and signature; compute original patient record attributes values;
b) transmit patient record ID, activity log, signature and original patient record attributes values to a data security provider;
c) store original patient record ID, activity log, signature and attributes values in digital storage;
d) provide patient record access to a user;
e) save new version of record, activity log and signature;
f) compute and save attributes values of new version of record;
g) transmit patient record ID, activity log, signature and attributes values of new version of record to Data Security Provider;
h) if the new attributes values same as original values then stop; otherwise proceed to the next step;
i) perform further analysis; and report findings.

### BRIEF DESCRIPTION OF THE DRAWINGS

FIG. 1 is an illustration of a typical healthcare enterprise comprising a healthcare provider, patients, practitioners, professionals, insurance providers, and digital data security provider, according to the preferred embodiment of the invention.

FIG. 2 is an illustration of a record, and information pertaining to the record such as identification, activity log, signature and attributes maintained in the computer storage, according to the preferred embodiment of the invention.

FIG. 3A is a flow diagram illustrating the method for authenticating a digital record utilizing certain attributes of the record according to a preferred embodiment of the invention.

FIG. 3B is a flow diagram illustrating another method for authenticating a digital record utilizing certain attributes of the record according to yet another preferred embodiment of the invention.

FIGS. 4A and 4B illustrate an example of detecting malicious tempering of a text record. FIGS. 4A and 4B illustrate an example of an original text record and the altered text record, respectively, and corresponding computed hash codes which are different.

FIGS. 5A and 5B illustrate an example of detecting malicious tempering of a 3D image. FIGS. 5A and 5B illustrate an example of an original image and the altered image, respectively, and the associated hash codes. The image shown is a three dimensional image of craniofacial features of a patient obtained by CT-scan.

FIGS. 6A and 6B illustrate an example of detecting malicious tempering of a speech record. FIGS. 6A and 6B illustrate an example of an original speech record and the altered speech record, respectively, displayed as wave diagrams, and the associated hash codes.

FIGS. 7A and 7B illustrate an example of detecting an error made in selecting 2D images. FIGS. 7A and 7B illustrate an example of an original 2D image and the 2D image selected through an error, respectively, and the associated hash codes. These images were taken through CT-scan.

FIGS. 8A and 8B illustrate an example of detecting a bona-fide change in 3D images. FIGS. 8A and 8B illustrate an example of an original 3D image of the dentition of a patient in malocclusion, and the 3D image of the dentition of the patient in the finished position, respectively, and the corresponding hash code values. Although the has hash codes are different indicating an image modification, it can be shown that the particular modification in the image is bona-fide.

### DETAILED DESCRIPTION OF PREFERRED EMBODIMENT

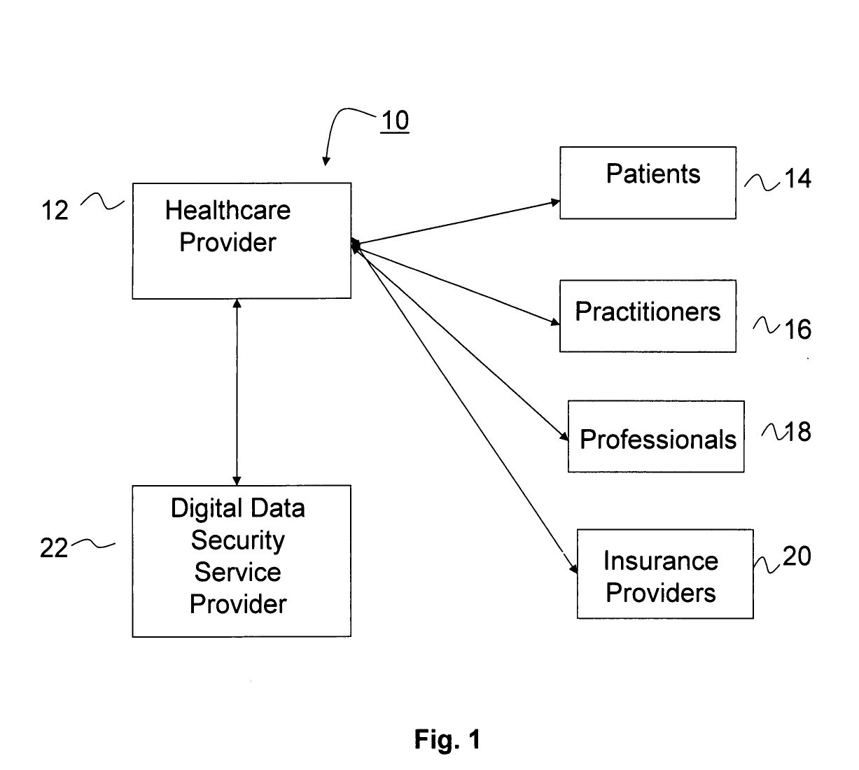

Before describing the invention, the background of the workings of the healthcare enterprise as it relates to the invention disclosed herein will be explained. FIG. 1 is an illustration of a typical healthcare enterprise 10 including a healthcare provider 12, such as for example a hospital. Patients 14 are referred to or come to the healthcare provider in order to receive healthcare. Practitioners 16 and professionals 18 are associated with the healthcare provider, and provide healthcare to the patients. Insurance providers 20 provide health insurance coverage to the patients; and therefore interact with the healthcare provider and the practitioners. Without loss of generality, the term insurance provider used here is inclusive of private companies as well as governmental agencies. In the process of providing the healthcare, the healthcare provider originates and maintains numerous records for each patient. The records carry a variety of information concerning patients. Typically, these records comprise text and images; and sometimes speech and video. Text records may include patient's diagnosis, treatment plan, prescription, billing, insurance and other information. Patient records may further include patient's living will, driving record and license, passport, educational records and other information deemed necessary for cross validation of patient identity. Images may be two-dimensional or three-dimensional; and may include photographs, x-rays, CT-scans, MRI, scanned images obtained from different types of scanners, and a variety of other images and models. Speech also may include patient's diagnosis, treatment plan, prescription, instructions to other healthcare professionals, etc. Some times digital videos are also included in patient records. It is increasingly becoming common in healthcare enterprises to store and communicate these records in digital form. These records are routinely accessed by a multitude of professionals and personnel in the healthcare industry, including off-shore practitioners in many instances, in order to deliver treatment and care to the patients. The records may be accessed by specialists for a peer-to-peer communication and consultation concerning healthcare issues of a patient. It is anticipated that the patient records may be modified by the practitioners or professionals as required during the planning and the administering phases of the treatment. Occasionally, the records may get modified through errors. However, it is also possible that the records may be tempered for malicious reasons. Therefore, in order to maintain complete integrity of patient records in the digital form, it is important to (a) detect when a record has been changed, and (b) distinguish the bona-fide record modification from errors and tempering. Digital data security service provider 22 provides methods and means for tracking the digital records based upon business rules established in consultation with the healthcare provider, or for that matter any subscriber of the security services provided by the data security provider. These may include providing different levels of security and access depending upon type and nature of record, use of record, the time over which access is allowed, the type of storage medium that the record may be copied to, the type of image analysis permitted; and detecting and identifying alterations in the records; thereby providing record authentication and forensic analysis services to the healthcare enterprise. Digital data security service provider may be an outside entity providing the services to the healthcare provider; or an entity integral to the healthcare provider. Furthermore, the digital record access and communications between the healthcare provider and practitioners, professionals, insurance providers, the digital data security service provider, and patients, when applicable, may be through Internet, Intra-net or a combination of Internet and Intra-net. One skilled in the art would appreciate that the communication medium could be any combination of land lines, fiber-optics, micro-wave, wireless and satellite based communications. One skilled in the art would appreciate that the state-of-the-art privacy, security and transmission error control measures offered by the telecommunications providers will be deployed in the digital records communications referred to herein.

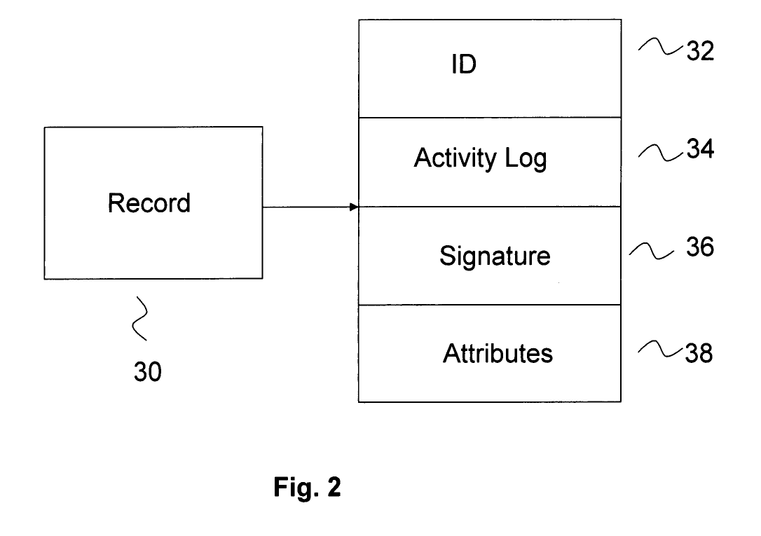

FIG. 2 is an illustration of a record 30, and information pertaining to the record maintained in the computer storage. Record 30 comprises the content of the patient record in the digital form, wherein the record may be it a text record, an image or a speech record. additionally, the record may be in the form of a digital video. The identification (ID) 32 comprises the patient name or a code uniquely associating the record with the patient, thereby enabling accurate access of the patient record by the practitioners and professionals. The ID may further comprise information such as, for example, the patient's birth date, address, etc. The activity log 34 provides information concerning creation and retrieval of the record. When the record is created for the first time, the activity log comprises, for example, the date and time the record preparation was started and completed; the location, including the device identification, where the record was created or produced, and any other information that may be pertinent. For any subsequent retrieval and storage of the record, the activity log comprises, for example, the date and time the record was opened and closed; the location including the device identification, where the record was accessed, and any other information that may be pertinent. In some instances, the activity log may simply comprise a time-stamp. The signature 36 comprises information, such as the user ID and password, identifying the person or persons who created the record, or person or persons who subsequently retrieved or accessed the record, which are typically required in controlling access of digital records. Additionally, the signature may comprise the person's one or more unique biometric identification data, such as a picture identification, finger print, voice signature, eye signature, etc. The voice signature may comprise words spoken at random according to a reference template which may be text-dependent or text-independent. One skilled in the art would appreciate that a normative database comprising signatures of all persons authorized to access the records is created and maintained by the healthcare provider and shared with the data security service provider, or created and maintained by the data security service provider alone. Additionally, associated with each record are its one or more attributes and their specific values 38. One such attribute is a hash code. The hash code is a hexadecimal value generated from a digitally stored file, which can be used as a means of ensuring that a file has not been tampered with or altered. The number of bits in a hash code depends on the algorithm used. The hash code is unique to the digital content of a record, irrespective of the type of the record. If the content is modified in any manner, the corresponding hash code changes accordingly. As noted earlier, the digital records of interest herein may comprise text, image or speech; and the hash code can be calculated for each of these types of records. There are numerous algorithms, e.g., MD5, SHA1, SHA512, RIPEMD160, available in prior art for calculating the hash codes of digital records. Another attribute of a text record is that, the differences, if any, between the first record and any other text record, which may be a version of the first record, can be identified by digitally comparing the contents of either selected parts, which may be selected at random, of the records or entire records. For an image record, a unique watermark can optionally be introduced in the image to serve as a unique attribute. The watermark may be visible or hidden from the view of the user. Additionally, one or more statistical random samples of pixels can serve as a unique attribute for an image record. Also, two image records can be compared pixel by pixel to identify the differences, if any, between the two image records. Similarly, the hash codes can be computed for text records and speech records as soon as they are prepared at the point of origin. A secret code using the inaudible range of the frequency of sound may be introduced in a speech record as an attribute. As for the video records, each frame can be treated as an image record; and the accompanying speech, if any, can be treated as a speech record. Furthermore, the image and video records may be encrypted using the prior art techniques.

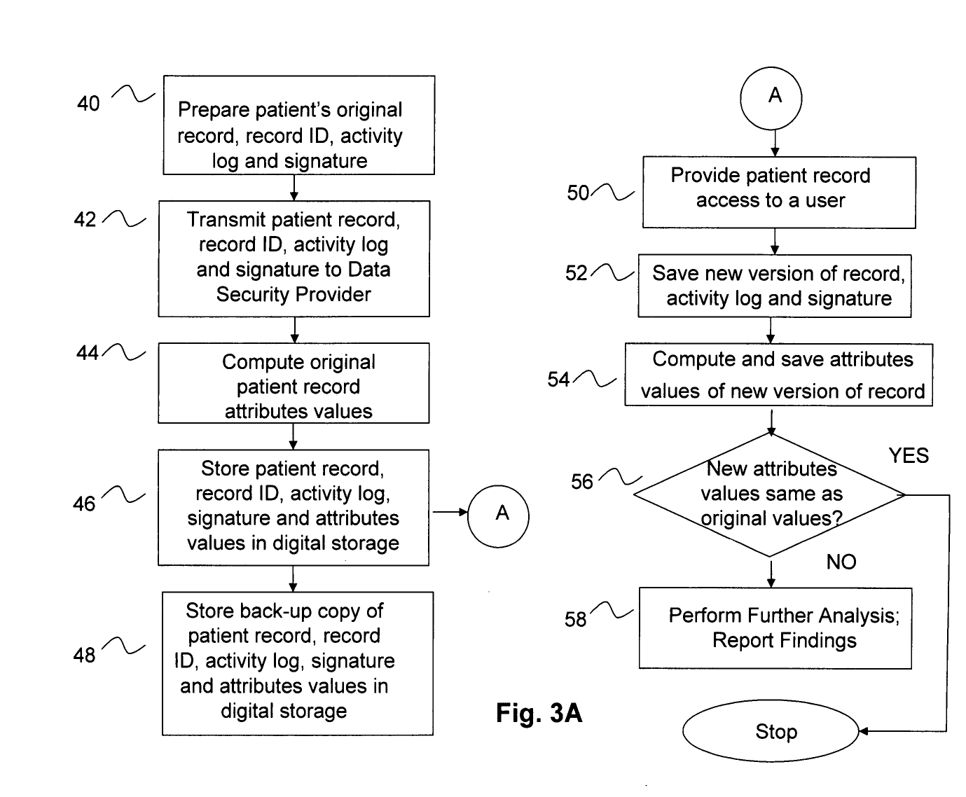

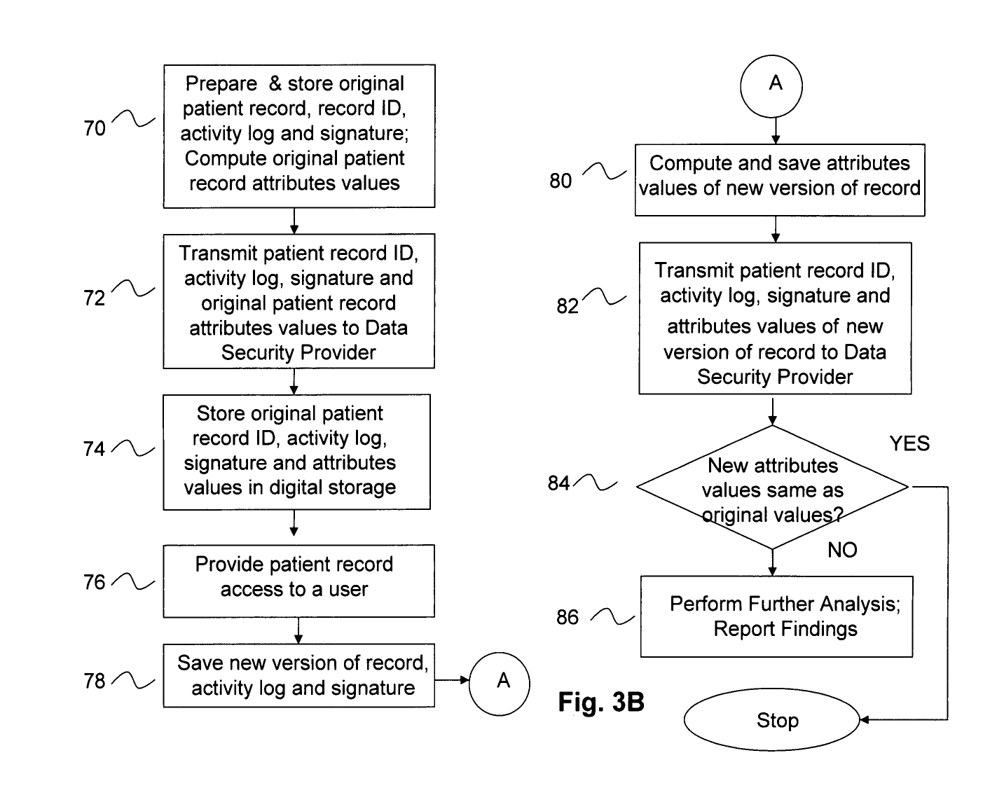

FIG. 3A is a flow diagram illustrating a novel service for authenticating digital records performed by a data security provider on behalf of a healthcare provider, according to the preferred embodiment of the invention. The process begins at step 40, where the digital record of a patient is created at a healthcare facility. In addition to the patient record, the record ID, the activity log, and the signature pertaining to the record are also created. Then, at step 42, the completed original digital record and associated information, such as the record ID, the activity log and the signature, are transmitted to the data security provider facility. Next, at step 44, the data security facility computes values of the one or more predefined attributes associated with the records; and at step 46 stores the record, the record ID, the activity log, the signature and the attributes values in a digital storage device located at the data security provider facility. Optionally, at step 48, a copy of the original patient record and the associated information such as the record ID, the activity log, the signature and the values of the attributes are stored in a back-up digital storage device for reliability purposes. Subsequently, at step 50, the data security provider provides accesses of the patient record to a user (i.e. permits opening of the record file) in accordance with the user specified record ID. The user may be a practitioner, a healthcare professional, or any one else authorized to access the record. The data security provider performs checks, and provides the access to the record once the user signature is validated. One skilled in the art would appreciate that the data security provider utilizes an enrollment database of signatures for the signature validation purposes. One skilled in the art would also appreciate that the signature verification and other mechanisms can be applied in order to assure proper access control. Furthermore, optionally, applicable business rules or operational protocol, such as the amount of time the record is allowed to be kept open, the frequency of access permitted to a user over a specified period of time, read-only access to some users, etc., may be applied by the data security provider in order to regulate and control the access to the record. Additionally, certain records may be encoded in a manner such that typically the records cannot be edited, e.g., text records encoded in certain pdf file format. Certain records may be allowed to be modified by additions only. Yet other records may be allowed to be edited by the designated individuals. When the user closes the record, the computer system located at the data security provider, at step 52, saves the patient's new version of the record and associated activity log and signature. Next, at step 54, values of the predefined attributes are computed for the new version of the record and saved. At step 56, the values of the attributes of the patient's original record are compared with the values for the new version of the record. If the attributes values are not changed, then the conclusion is that the original version of the record is not modified; and the authentication process is completed. On the other hand, if the attributes values for the new version of the record are different from the attributes values for the original version of the record, then it indicates that modifications in the original record have been made. In that case, at step 58, further analysis is made to identify and evaluate the nature of the modifications. One skilled in the art would appreciate that steps 54 and 56 may simply examine a selected predefined attribute, such as hash code. In that case, further analysis may comprise computation and comparison of additional predefined attributes in any preferred sequence. Additional analysis may comprise comparison of entire records or a selected portion thereof. For example, for the digital image records, the predefined attributes may include one or more statistically or otherwise selected samples of pixels. Further analysis may include identification of the modification and evaluation of the significance or validity of the modification. One skilled in the art would appreciate that the significance of a particular modification may be judged by applying a set of predefined rules. For example, the change in the background color in an image may not have any significance; but deletion of a portion of an image, such as tumor, may be very significant. Further analysis may comprise evaluating the significance of a change in the record by comparing the change with the acceptable range of values set forth in applicable standard references. For example the strength for a particular drug prescribed to a patient was specified at one value in the original prescription, and changed to another value in the modified record under review. The significance of the modified prescription can be evaluated automatically using a computer by comparing the modified does with the does recommended in the healthcare industry standard Prescription Drug Reference (PDR) or any other authoritative source accepted by the industry. One skilled in the art would appreciate that the scope for further analysis of this nature is not meant to be limited to the types of evaluations described herein. The results of the record modification analysis are subsequently reported by the data security provider to the healthcare provider. In the healthcare enterprise, it is understood and expected that the patient records may be justifiably modified by the practitioners and/or professionals as deemed necessary for delivering health care to the patients. So, the challenge is to catch the record alterations that amount tempering. The invention disclosed herein provides a novel method and system comprising a service business practice for authentication of patient records by (a) detecting one or more modifications made to a patient's original record, (b) evaluating the significance of the one or more modifications thereby filtering out bona fide modifications and (c) subjecting suspicious modifications to detailed scrutiny thereby isolating tempering.

According to one embodiment of the invention, the data security provider provides centralized digital storage devices and facility for digitally storing all versions of the patient records and associated information discussed above such as the record ID, the activity log, the signature and the computed values of the one or more preferred attributes. The records and the information is archived after a certain time period, and preserved for another certain time period mutually agreed upon between the health care provider or any service subscriber and the data security provider.

According to another embodiment of the invention, the data security provider provides distributed digital storage devices and facility for digitally storing all versions of the patient records and associated information discussed above. One skilled in the art would appreciate that a combination of the centralized storage for some applications, and the distributed storage for others can very well be realized.

FIG. 3B is a flow diagram illustrating a novel service for authenticating digital records performed by a data security provider on behalf of a healthcare provider, according to another preferred embodiment of the invention. The process begins at step 70, where the digital record of a patient is created at a healthcare facility. The record ID, the activity log, and the signature pertaining to the hardware and record are also created. Additionally, the health care facility computes values of the one or more predefined attributes associated with the record. Preferably, the device used for creating the record is set-up to calculate the values of the one or more preferred attributes. The health care facility digitally stores, in one or more computer storage devices, the patient original record, the record ID, the activity log, the signature pertaining to the record, and the values computed for the one or more preferred attributes of the record. Optionally, a copy of the original patient record and the associated information such as the hardware ID, record ID, the activity log, the signature and the values of the one or more pre-selected attributes are stored in one or more back-up digital storage device at the health care facility for reliability purposes. Then, at step 72, the information associated with the completed original digital record, such as the record ID, the activity log, the signature and the values computed for the one or more preferred attributes of the record, are transmitted to the data security provider facility, where, at step 74, they are stored for subsequent use in authenticating future versions of the record. Subsequently, at step 76, the health care provider provides accesses of the patient record to a user (i.e. permits opening of the record file) in accordance with the user specified record ID. Indeed, in some instances, a copy of the record may be transmitted to another facility associated with the health care facility for enabling the access to the record. The user may be a practitioner, a healthcare professional, or any one else authorized to access the record. The health care provider performs checks, and provides the access to the record once the user signature is validated. One skilled in the art would appreciate that the health care provider utilizes an enrollment database of signatures for the signature validation purposes. One skilled in the art would also appreciate that the signature verification and other mechanisms can be applied in order to assure proper access control. Furthermore, optionally, applicable business rules or operational protocol, such as the amount of time the record is allowed to be kept open, the frequency of access permitted to a user over a specified period of time, read-only access to some users, etc., may be applied by the health care provider in order to regulate and control the access to the record. Additionally, certain records may be encoded in a manner such that typically the records cannot be edited, e.g., text records encoded in certain pdf file format. Certain records may be allowed to be modified by additions only. Yet other records may be allowed to be edited by the designated individuals. When the user closes the record, the computer system located at the health care provider, at step 78, saves the patient's new version of the record and associated activity log and signature. Next, at step 80, values of the predefined one or more attributes are computed for the new version of the record and saved at the facility from where the record was accessed. At step 82, the patient record ID, the activity log, the signature and values of the one or more predefined attributes corresponding to the new version of the record are digitally transmitted to the data security provider facility. At step 84, the values of the one or more preferred attributes of the patient's original record are compared with the values for the new version of the record, wherein the comparison is performed at the data security provider facility. If the attributes values are not changed, then the conclusion is that the original version of the record is not modified; and the authentication process is completed. On the other hand, if the attributes values for the new version of the record are different from the attributes values for the original version of the record, then it indicates that one or more modifications in the original record have been made. In that case, at step 86, further analysis is made to identify and evaluate the nature of the modifications. Here again, one skilled in the art would appreciate that steps 80, 82 and 84 may simply examine a selected predefined attribute, such as hash code. In that case, further analysis may comprise computation, transmission and comparison of additional predefined attributes in any preferred sequence with collaboration between the data security provider facility and the health care provider facility. As discussed earlier with respect to FIG. 3A, here again the additional analysis may comprise comparison of entire records or a selected portion thereof. In this case the new version of the record is also transmitted to the data security provider facility so as to enable the further analysis. For example, for the digital image records, the predefined attributes may include one or more statistically or otherwise selected samples of pixels. Further analysis may include identification of the modification and evaluation of the significance or validity of the modification. One skilled in the art would appreciate that the significance of a particular modification may be judged by applying a set of predefined rules. For example, the change in the background color in an image may not have any significance; but deletion of a portion of an image, such as tumor, may be very significant. Further analysis may comprise evaluating the significance of a change in the record by comparing the change with the acceptable range of values set forth in applicable standard references. For example the strength for a particular drug prescribed to a patient was specified at one value in the original prescription, and changed to another value in the modified record under review. The significance of the modified prescription can be evaluated automatically using a computer by comparing the modified does with the does recommended in the healthcare industry standard Prescription Drug Reference (PDR). One skilled in the art would appreciate that the scope for further analysis of this nature is not meant to be limited to the types of evaluations described herein. The results of the record modification analysis are subsequently reported by the data security provider to the healthcare provider or the subscriber of the service. In the healthcare enterprise, it is understood and expected that the patient records may be justifiably modified by the practitioners and/or professionals as deemed necessary for delivering health care to the patients. So, the challenge is to catch the record alterations that amount to tempering. The invention disclosed herein provides a novel method and system comprising a service business practice for authentication of patient records by (a) detecting one or more modifications made to a patient's original record, (b) evaluating the significance of the one or more modifications thereby filtering out bona fide modifications and (c) subjecting suspicious modifications to detailed scrutiny thereby isolating tempering.

In another aspect of the invention, although the preceding discussion focused on comparing the original version of a patient record with a subsequent version of the patient record; one skilled in the art would appreciate that the method can be generalized for comparing any two versions of the record.

In yet another aspect of the invention, the method can similarly be applied for authentication of any type of records, and in any type of business; such as for example financial records in financial institutions.

In yet another aspect of the invention, the method can be applied for authentication of books and manuscripts or pieces of art kept in the digital form.

In order to further illustrate the concepts of the instant invention, several figures will now be explained.

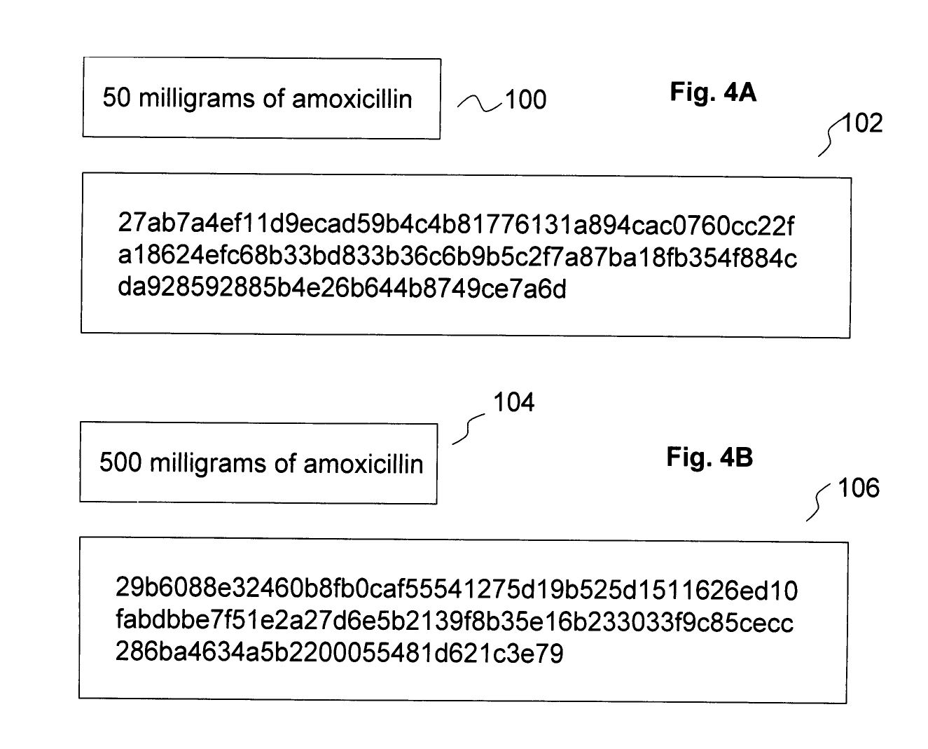

FIGS. 4A and 4B illustrate an example of detecting malicious tempering of a text record. FIG. 4A illustrates an example original text record 100 and the corresponding computed value 102 of the hash code; and FIG. 4B the altered text record 104 and the corresponding computed value 106 of the hash code vale. As can be seen from FIG. 4A, the text record 100 reads “50 milligrams of amoxicillin,” which is a prescription for a patient. The text record was subsequently altered as record 104 in FIG. 4B; and the hash code 106 in FIG. 4B was computed for the text record 104. Upon comparison of the hash code 102 of the original record 100 with the hash code 106 of the modified record 104, it was found that the value of the new hash code was not the sane as the value of the original hash code, thereby indicating that the record was modified. Subsequently, the entire original record was compared with the new record; and it was confirmed that the prescription strength was changed from 50 milligrams to 500 milligrams. The new prescription does was compared with the recommendations in PDR, and it was determined that the new does was not recommended for the illness of the patient. So, in this manner, the record tempering can be detected and brought to the attention of the healthcare provider with necessary evidence for further action.

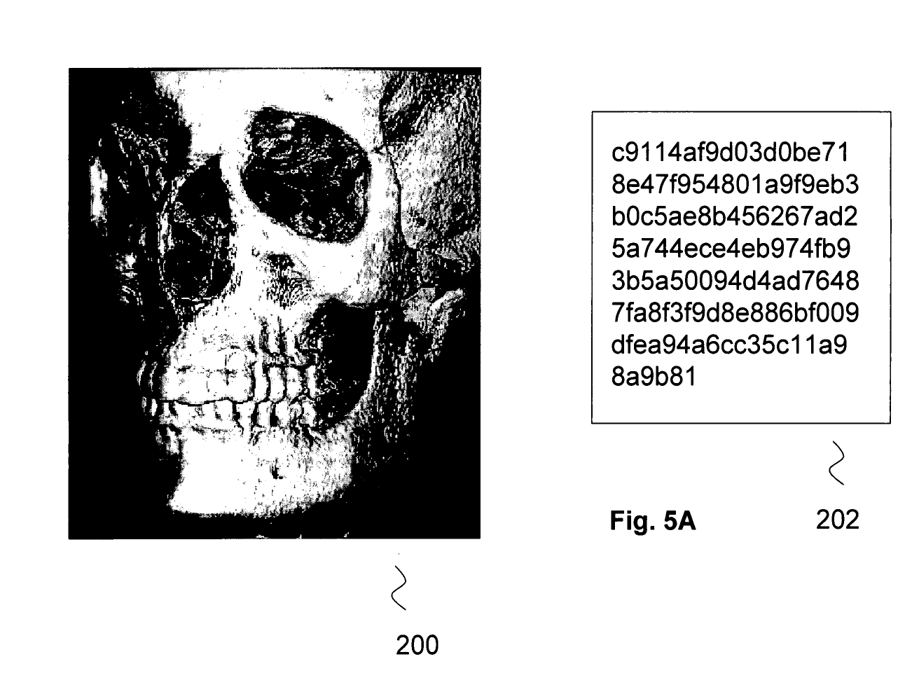

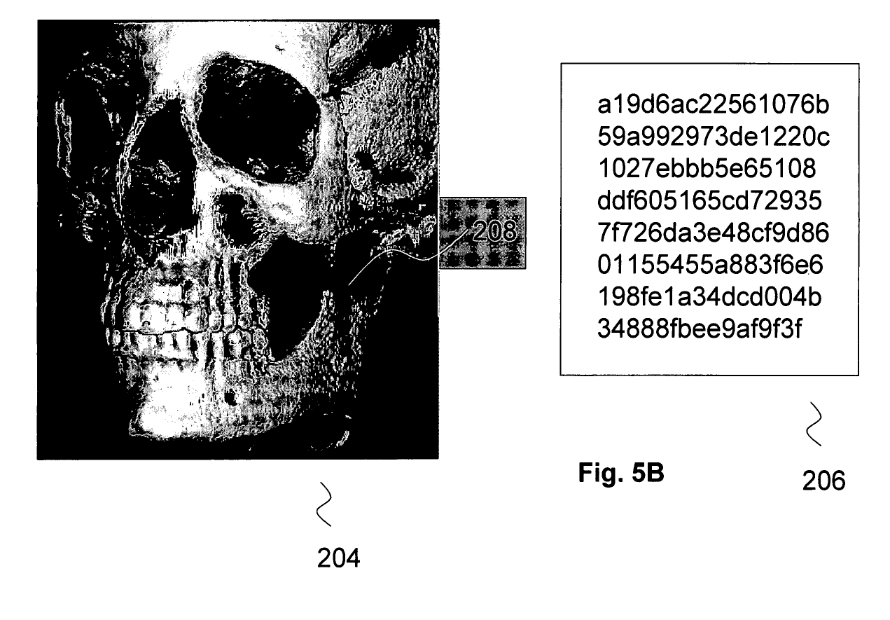

Similarly, FIGS. 5A and 5B illustrate an example of detecting malicious tempering of a 3D image record. FIGS. 5A and 5B illustrate an example of an original image and the altered image, respectively, and associated hash codes. The image is a three dimensional image of craniofacial features of a patient obtained by CT-scan. FIG. 5A illustrates an example original 3D image 200, and the associated hash code 202. The image was subsequently altered as image 204 in FIG. 5B, and the corresponding hash code 206 in FIG. 5B was computed for the text record 204. Upon comparison of the hash code 202 of the original record 200 with the hash code 206 of the record 204, it was found that the value of the new hash code was not the sane as the value of the original hash code, thereby indicating that the record was modified. A portion 208 was removed from the original image 200, thereby producing the tempered image 204.

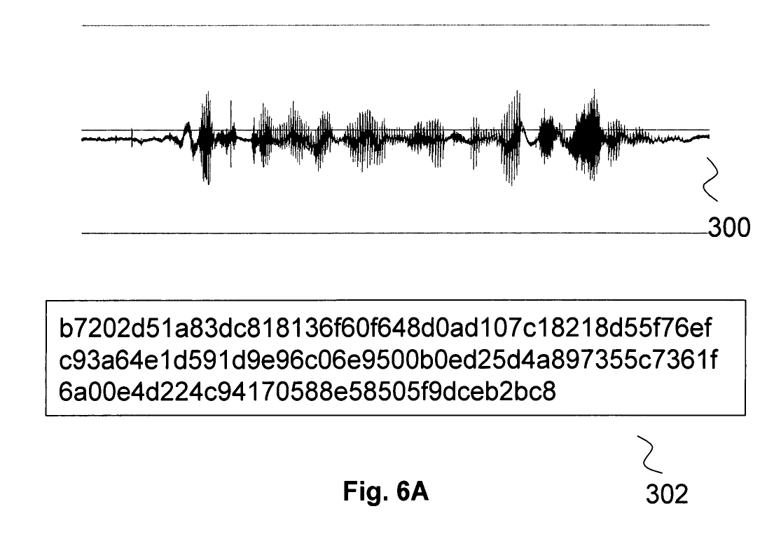

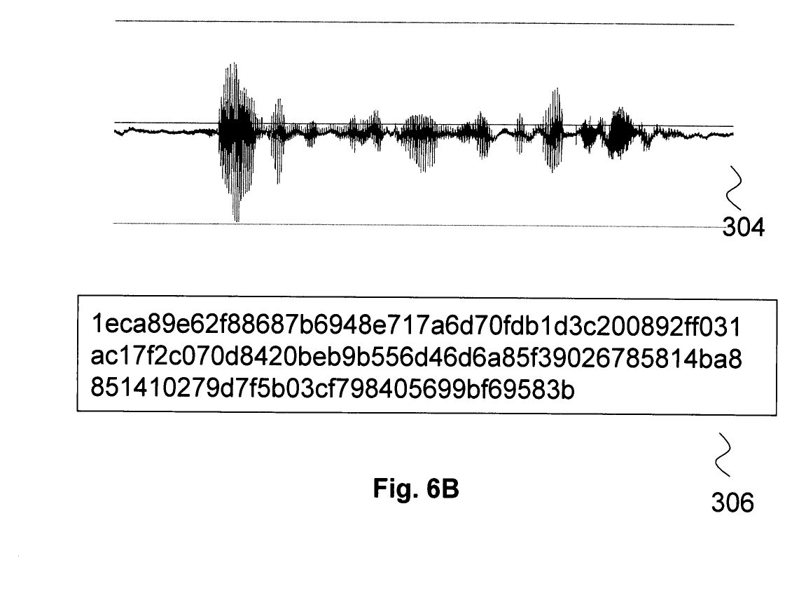

Similarly, FIGS. 6A and 6B illustrate an example of detecting malicious tempering of a speech record. FIGS. 6A and 6B illustrate an example of an original speech record and altered speech record, respectively, displayed as wave diagrams, and associated hash codes. FIG. 6A illustrates an example original wave diagram 300 of an original speech record, and the associated hash code 302. The speech record was subsequently altered as shown as the wave-form 304 in FIG. 6B, and the hash code 306 in FIG. 6B was computed for the speech record 304. Upon comparison of the hash code 302 of the original record 300 with the hash code 306 of the record 304, it was found that the value of the new hash code was not the sane as the value of the original hash code, thereby indicating that the speech record was modified. Subsequently, the entire original record was compared with the new record; and it was confirmed that the prescription strength was changed from 50 milligrams to 500 milligrams.

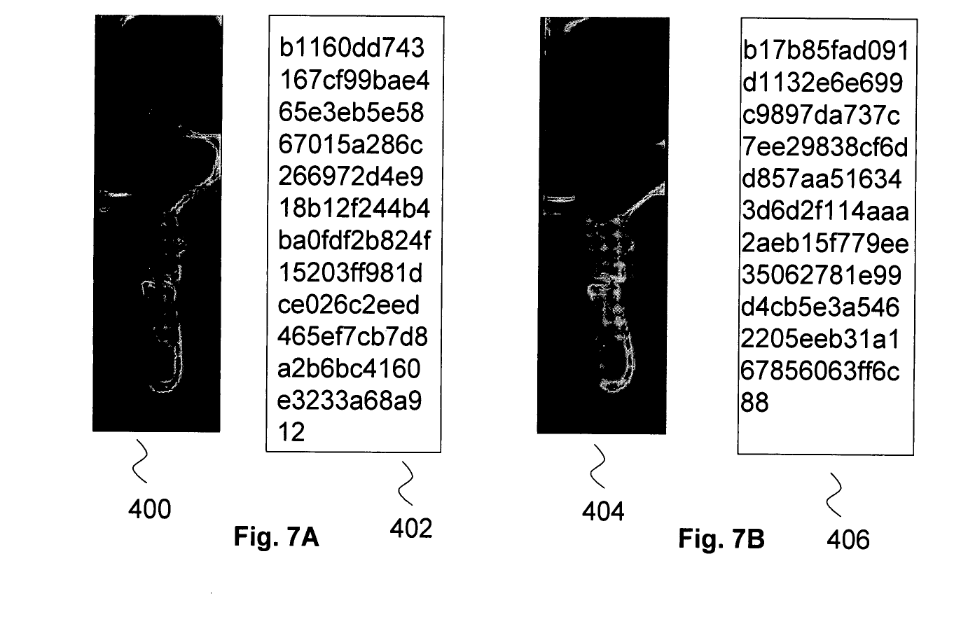

FIGS. 7A and 7B illustrate an example of detecting an error made in selecting 2D images. FIGS. 7A and 7B illustrate an example of an original 2D image and the 2D image selected through an error, respectively, and the associated hash codes. These images were taken through CT-scan. FIG. 7A illustrates a 2D image slice 400 of a tooth. FIG. 7B on the other hand illustrates a 2D image of another slice 404 of the same tooth. The slice 404 was chosen by mistake. So even though the hash code 406 value for the image 404 is different than the hash code value 402 for the image 400; through further analysis it can be shown that the image 404 was the result of an error in selecting the proper image rather that tempering with the original image 400.

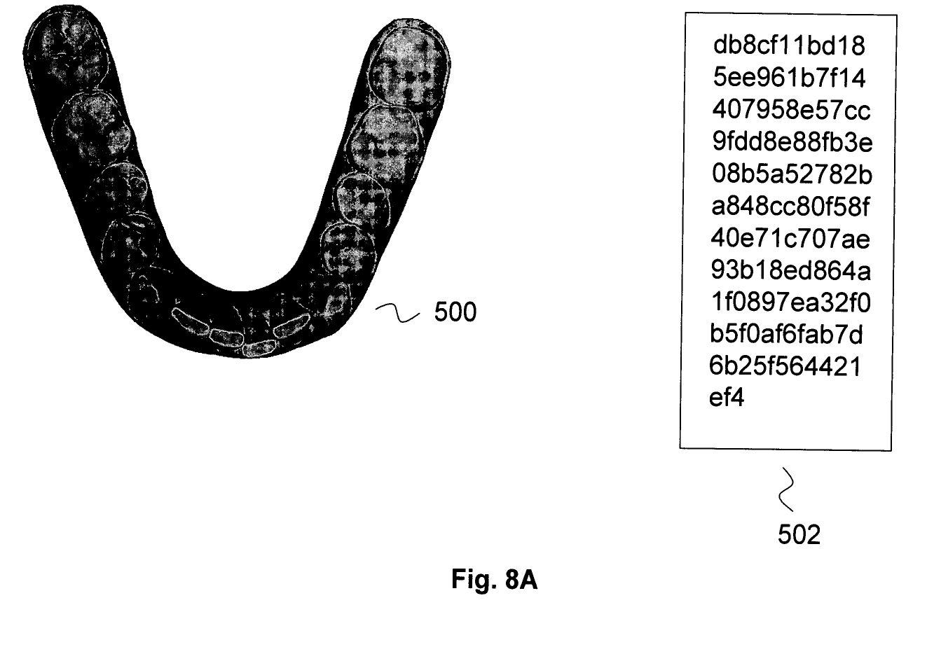

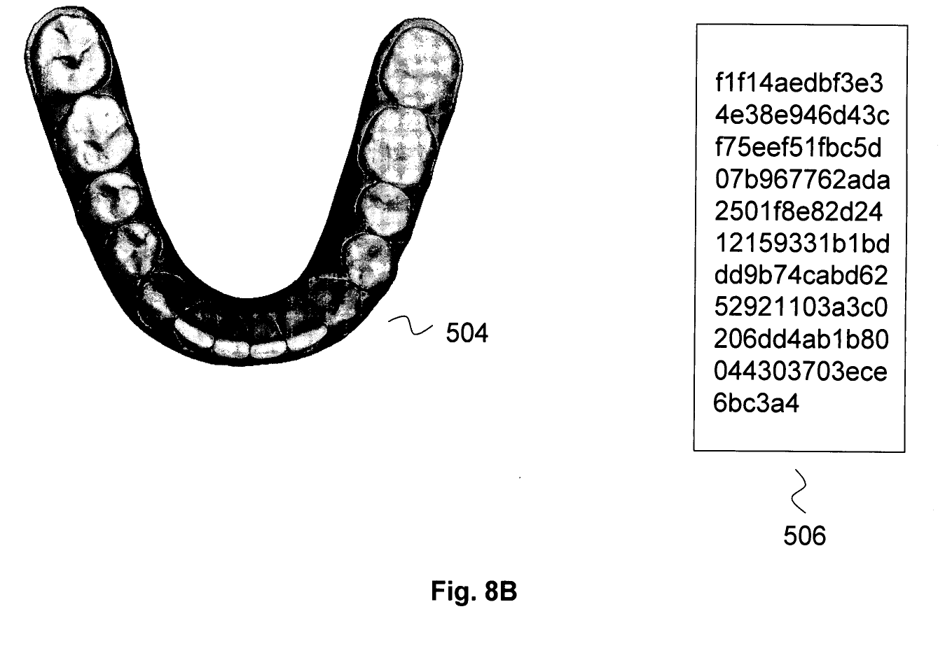

FIGS. 8A and 8B illustrate an example of detecting a bona-fide change in 3D images. FIGS. 8A and 8B illustrate an example of an original 3D image 500 of the dentition of a patient in malocclusion, and the 3D image 504 of the dentition of the patient in the finished position, respectively, and the corresponding hash code values 502 and 506. Although the hash codes 502 and 506 are different indicating an image modification, it can be shown that the particular modification in the image is bona-fide.

One skilled in the art would appreciate that all different types of images discussed above are stored in digital form in the electronic storage devices accessably coupled with one or more digital computing devices.

For the sake of brevity, the digital file corresponding to each of the records discussed above is not shown herein. The hash code for each record discussed above was computed using the SHA-512 algorithm.

In another aspect of the invention, the authentication process disclosed herein can be modified for conducting a forensic analysis in the event that a patient record has been determined to be tempered with in an unjustifiable manner. Since all versions of the records and the information associated with the records are digitally stored and maintained between the health care provider and the data security provider; the entire history of the records can be traced in support of the forensic analysis.

In yet another aspect of the invention, the method can be used for authenticating, for example, the prescribed medicine being carried by an airline passenger. The authentication process would work as follows:
(a) An official at the airport scans the prescription label on the medicine bottle being carried by a passenger, along with a picture identification of the passenger; and transmits the information digitally to a data security provider;
(b) The data security provider maintains, and continually updates, a database of the valid prescriptions and the picture identifications of the patients;
(c) The data security provider compares the information received from the airport official with the information in the database; and
(d) (i) If the information completely matches, then the data security provider sends a ‘green signal’ to the air port official;
   (ii) If the information matches partially, then the data security provider sends a ‘yellow signal’ to the air port official; suggesting that further investigation may be required; and
   (iii) If the information does not match at all, then the data security provider sends a ‘red signal’ to the air port official; suggesting that the authentication has failed.

In another aspect, a system for authentication of digital records is disclosed. The system comprises one or more workstations, each having a processor and a storage device which stores patient records in digital form in healthcare enterprises. The digital records may be in the form of text, images, video or speech. The digital images may be two-dimensional or three-dimensional comprising, for example, photographs, x-rays, CT-scans, other types of scanned images, video, etc. Additionally, the system provides computer instructions, which are stored in one or more digital storage devices enabling the user to:

a) prepare or receive patient's original record, record ID, activity log and signature;

b) transmit patient record, record ID, activity log and signature to a data security provider;

c) compute original patient record attributes;

d) store patient record, record ID, activity log, signature and attributes in digital storage;

e) store back-up copy of patient record, record ID, activity log, signature and attributes in digital storage;

f) provide patient record access to a user;

g) save new version of record, activity log and signature;

h) compute and save attributes of new version of record;

i) if new attributes are the same as the original attributes, then stop; otherwise proceed to the next step;

j) perform further analysis; and report findings.

In another aspect of the invention, a system for record authentication is disclosed comprising one or more workstations, each having a processor and a storage device which stores patient records in digital form in healthcare enterprises. The digital records may be in the form of text, images, video or speech. The digital images may be two-dimensional or three-dimensional comprising, for example, photographs, x-rays, CT-scans, other types of scanned images, video, etc. Additionally, the system provides computer instructions, which are stored in one or more digital storage devices enabling the user to:
a) prepare or receive & store original patient record, record ID, activity log and signature; compute original patient record attributes values;
b) transmit patient record ID, activity log, signature and original patient record attributes values to a data security provider;
c) store original patient record ID, activity log, signature and attributes values in digital storage;
d) provide patient record access to a user;
e) save new version of record, activity log and signature;
f) compute and save attributes values of new version of record;
g) transmit patient record ID, activity log, signature and attributes values of new version of record to Data Security Provider;
h) if the new attributes values same as original values then stop; otherwise proceed to the next step;
i) perform further analysis; and report findings.

In one embodiment of the invention copying of the records is restricted to authorized persons only, which may be under specified rules, and may further restrict the media on which the copy can be made.

In yet another embodiment of the invention, the services of the data security provider disclosed above are extended to the service subscribing patients; who are then permitted to access their own records.

One skilled in the art would appreciate that, regarding speech records, additional techniques such as speaker verification and or speaker identification can be employed. The speaker verification deals with determining if a speaker is really who he/she claims to be. On the other hand, the speaker identification deals with matching a person to the speech record. Both of these techniques use a stored database of reference templates for known speakers and employ similar speech analysis and decision techniques. Speech imposters can be identified using these techniques. Speech recognition techniques can also be used to automatically determining the content of a person's speech. Additionally, speech-to-text or speech dictation systems may be used in order to transcribe speech to text. Also, if need be, an automatic language identification technique may be used for identifying the language spoken by a person regardless of content of the speech.

In yet another aspect, a method of conducting forensic analysis of tempered digital records is disclosed. In summary, the forensic analysis comprises:

(a) digitally storing all versions of records;

(b) examining record history and records;

(c) identify record changes, location and time; and

(e) evaluating the record modifications and identifying the sources.

Additionally, the forensic analysis may optionally require initializing all hardware components in the authentication system.

Persons skilled in the art will appreciate that variation from the details of the presently preferred and alternative embodiments may be made without departure from the true scope and spirit of the invention. The true scope is to be determined by reference to the appended claims.

We claim:

1. A method for providing service by a data security provider for authentication of digital records in healthcare enterprise, comprising the following steps:
a) preparing a patient's original record, record ID, activity log and signature at the health care provider facility;
b) transmitting the patient's original record, record ID, activity log and signature to the data security provider;
c) computing original patient record attributes;
d) storing the patient's original record, record ID, activity log, signature and attributes in a digital storage at the data security provider facility;
e) providing the patient's record access to a user;
f) saving new version of the patient's record, activity log and signature;
g) computing and saving the attributes of the new version of the patient's record;
h) comparing the attributes of the new version of the patient's record with the attributes of the original version of the record; and
i) if new attributes are not the same as the original attributes, then performing further analysis of the new version of the record, and reporting the findings to the healthcare provider.

2. The method of claim 1, wherein the digital records are in the form of text.

3. The method of claim 1, wherein the digital records are in the form of an image.

4. The method of claim 1, wherein the digital records are in the form of video.

5. The method of claim 1, wherein the digital records are in the form of speech.

6. The method of claim 1, wherein the digital records include the patient's living will.

7. The method of claim 6, wherein the patient's living will is made accessible to one or more practitioners treating the patient; and to one or more relatives of the patient.

8. A method for providing service by a data security provider for authentication of digital records in healthcare enterprise, comprising the following steps:
a) preparing and storing a patient's original record, record ID, activity log and signature; and computing and storing attributes of the patient's original record at the health care provider facility;
b) transmitting the patient's original record ID, activity log, signature and the attributes of the patient's original record to the data security provider;
c) storing the patient's original record ID, activity log, signature and the attributes of the patient's original record in a digital storage at the data security provider facility;
e) providing the patient's record access to a user;
f) saving new version of the patient's record, activity log and signature at the healthcare provider facility;
g) computing and saving the attributes of the new version of the patient's record at the healthcare provider facility;
h) transmitting the patient's record ID, activity log, signature and the attributes of new version of the patient's record to the data security provider;
h) comparing the attributes of the new version of the patient's record with the attributes of the original version of the record; and
i) if new attributes are not the same as the original attributes, then performing further analysis of the new version of the record, and reporting the findings to the healthcare provider.

9. The method of claim 8, wherein the digital records are in the form of text.

10. The method of claim 8, wherein the digital records are in the form of an image.

11. The method of claim 8, wherein the digital records are in the form of video.

12. The method of claim 8, wherein the digital records are in the form of speech.

13. The method of claim 8, wherein the digital records include the patient's living will.

14. The method of claim 13, wherein the patient's living will is made accessible to one or more practitioners treating the patient; and to one or more relatives of the patient.

15. A system for authentication of digital records, comprising:
one or more workstations, each having a processor operably coupled to one or more storage devices;
wherein the storage devices store patient records in a digital form in the healthcare enterprise; and wherein the one or more storage devices provides computer instructions enabling an user to:
a) prepare or receive patient's original record, record ID, activity log and signature;
b) transmit the patient's original record, record ID, activity log and signature to a data security provider;
c) compute attributes of the original patient record;
d) store patient record, record ID, activity log, signature and attributes in digital storage;
e) provide the patient's record access to a user;
f) save new version of record, activity log and signature;
g) compute and save the attributes of new version of record;
h) compare the attributes of new version of record with the attributes of original version of the record; and
i) if the new attributes are not the same as the original attributes, then perform further analysis of the new version of the record; and report the findings to the healthcare provider.

16. The system of claim 15, wherein the digital records are in the form of text.

17. The system of claim 15, wherein the digital records are in the form of an image.

18. The system of claim 15, wherein the digital records are in the form of video.

19. The system of claim 15, wherein the digital records are in the form of speech.

20. The system of claim 15, wherein the digital records include the patient's living will.
